(19) United States
(12) Patent Application Publication
(10) Pub. No.: US 2023/0226106 A1
(43) Pub. Date: Jul. 20, 2023

(54) A Novel formulation for rapid wound healing and control of infection

(71) Applicant: Milind Choudhari, (US)

(72) Inventor: Milind Choudhari, (US)

(21) Appl. No.: 16/310,423

(22) PCT Filed: Jul. 22, 2017
(86) PCT No.: PCT/IN2017/005301

§ 371 Date: Dec. 15, 2018

(51) Int. Cl. A61K 33/38; A61K 31/688; A61K 31/05; A61P 17/02; B82Y 5/00

(52) U.S. Cl. CPC A61K 33/38; A61K 31/688; A61K 31/05; A61P 17/02; B82Y 5/00

(57) ABSTRACT

The present invention is a formulation of organic and non-organic components for rapid wound healing and control of infection. The components of the present invention i.e. the formulation are an antimicrobial agent, an antioxidant, a cell proliferation inducing factor and an organic component responsible for matrix formation. Adding these four components in pre-determined proportions, formulations can be made for gels, liquid solution and biodegradable scaffolds which are safe and gives a synergistic effect.

### FIELD OF INVENTION

Present invention relates to a novel organic and inorganic nano-formulation for antimicrobial activity and wound healing. More particularly the invention relates to a formulation of, antibacterial agent, antioxidant agents and an inducer of endothelial cell proliferation to be used in gel, cream, spray, liquid solution and bandages for wound care.

### BACKGROUND OF INVENTION

Critical wound are difficult to heal or sometimes non-healing, leading to amputation. It is making 48,000 people disabled in India.

Wound care is one of the major factors in controlling the infection. Today when the conventional antibiotics are used for wound care there is a constant threat of antibiotic resistance. Moreover, such wound dressings are manufactured with a sole goal of preventing infections. There is great need of an alternative to the conventional way of using antibiotics in wound dressing, which will aid in better and faster wound healing with the help of added components with beneficial activities.

The management of wound varies according to the category of the wounds, which includes diabetic wounds, pressure wounds/ulcers; venous ulcers, skin burns wounds, post traumatic ulcers, and general surgery.

Amongst these diabetic foot ulcers, pressure wounds and venous ulcers are considered as critical wounds and result into necrosis, impaired vascularization, improper fibroblast formation and alleviated MMP2 levels.

There are numerous topical regimens and devices available for the management of diabetic foot wounds including hydrogels, hydrocolloids, alginates, foam, silver impregnated dressings, growth factors, silicon impregnated atraumatic dressings, vacuum aided devices, hyperbaric oxygen therapy, etc. However, before choosing a regime one should consider factors such as the general health of the patient, the process of tissue repair, assessment of the wound by means of grading, description and classification of the wound, local environment of the wound, knowledge on specific properties of the dressing materials and devices as well as their availability, affordability, and accessibility.

Management and treatment options available for such wounds are:

1^{st} line of treatment:
Debridement
Hyperbaric Oxygen Chamber
Negative Pressure Therapy

2^{nd} line of treatment:
Bandages
Hydrochlorides
Hydrogels
Sponges

3^{rd} line of treatment:
Collagen
Skin substitute
Xenograft

4^{th} line of treatment
Growth factors such as PDGF

Wound healing is a complex process involving highly regulated responses of specified cell types, which harbor locally secreted growth factors that play a key role in wound healing.

U.S. Pat. No. 8,686,051 B2 discloses a pharmaceutical composition for treatment of diabetic complications by administration of beta-blockers. The invention further discloses employing a topical formulation of a beta-blocker, having substantially no antibacterial activity, to improve the process of diabetic wound healing. The composition as disclosed in the cited patent takes care of the diabetic wound and is responsible only for wound healing.

The tools and techniques available offer potential recovery rates along with some drawbacks are observed. These drawbacks are toxicity, in case of use of silver impregnated dressings, lack of vascularization due to improper penetration of PDGF at the wound site, antibiotic resistance due to extensive use of antibiotics, increase rate of nosocomial infection due to microorganisms such as Staphylococcus aureus, Methicillin resistant Staphylococcus aureus, Escherichia coli, frequent dressings i.e. opening and closing of wound for dressings in hospitals.

There is great need of an alternative to the conventional way of using antibiotics in wound dressing, which will aid in better and faster wound healing with the help of added components with beneficial activities. The formulation of the present invention is a mixture of organic and inorganic components and provides antibacterial effect, safeguard from reactive oxygen species (ROS) as well as rapid wound healing properties.

### OBJECTS OF THE INVENTION

The primary object of the invention is to develop an organic and inorganic nano-formulation for rapid wound healing and antimicrobial activity.

Another object of the present invention is to develop an organic and inorganic nano-formulation having a synergistic effect of Gallic Acid, Sphingosine-1-Phosphate, Silver nanoparticles

Yet another object of the present invention is to eradicate the need of amputation in turn minimizing the disability.

Another object invention is to reduce the toxicity of silver, achieved by addition of Gallic acid which masks the silver toxicity.

Still another object of the invention is to develop an organic and inorganic nano-formulation is to make it into liquid solution, cream/gel and biodegradable scaffolds.

Another object of invention is the formulation using antibacterial, antioxidant and cell proliferating molecules for rapid healing of wounds.

### SUMMARY OF THE INVENTION

The formulation of the present invention provides a holistic solution to issues associated with wound healing and consists of 3 components organic (Sphingosine 1 phosphate), inorganic (Silver nanoparticles) and an antioxidant agent (Gallic Acid) and provides a nano-formulation for rapid wound healing and control of infection. The formulation is developed in such a way that the mixing the components in appropriate proportions gives an antimicrobial and wound healing effect in a synergistic way. Additionally the composition also helps in faster wound healing by the way of inducing the cell proliferation, blood vessel formation and collagen matrix formation.

Each of the above component has been addressed with a specific role in wound healing. Silver nanoparticles act as effective antibacterial agent at the wound site. The key molecule Sphingosine-1-Phosphate is a lipid molecule, which helps in proliferation of the endothelial cells required at the wound area. Gallic acid is used to reduce the oxidative stress at the wound area.

### DETAILED DESCRIPTION OF THE INVENTION

The present invention discloses an organic and inorganic nano-formulation for rapid wound healing and control of infection. The said compositions are designed to offer improved advantages with respect to wound healing along with control of infection. The nano-formulation is prepared by adding sphingosine-1-phosphate (10 μM-1 mM/g of gel), Silver Nanoparticles (0.8 mg/g-1.5 mg/g of gel), Gallic acid (1 mg/g-5 mg/g of gel)

In order to confirm the activity of present formulation different studies were performed viz. histopathological examination, immunohistochemistry, ROS analysis, hematology and molecular expression study.

### Immunohistochemistry:

To visualize Ki67 expression in wound skin sections, formalin-fixed paraffin-embedded tissue are cut in sections of 5-7 μm thickness. The sections are deparaffinized, blocked, and incubated with monoclonal Ki-67 primary antibody (1:50 dilution) for 1 h. The sections are then incubated with HRP-IgG (1:200 dilution) for 1 h, followed by incubation with 3,3′-diaminobenzidine hydrochloride substrate for 30 min in the dark. Cells were counterstained using hematoxylin (1:50 dilution in distilled water) for 1 h. Slides were then imaged with an optical microscope. For semi quantitation, the Ki67 cells were counted from three different fields across all groups.

### Histopathological Analysis:

Histopathological analysis of the skin at wound site is performed for healing process with presence of inflammatory cells, neutrophils, macrophages, proliferation of granulation tissue comprised of fibroblasts and neovascularization process and collagen deposition in the healing process.

The inflammatory cells neutrophils and macrophages were more in diseased control as compared with the standard control and the current formulation. The presence of inflammatory cells confirms prolong inflammation in control group which delays wound healing. The presence of these cells shows an early stage of wound healing in diseased control rats while in the rats treated with the current formulation the inflammation had reduced indicating the progress of wound healing. Granulation tissue, proliferation of fibroblast cells and Collagen deposition was more in the current formulation than in control and standard gel.

The Collagen deposition was more compact in the group of rats treated with the current formulation. In the control group the deposition is in patches and not in a compact manner.

Collagen is an important protein produced by fibroblast cells and helps in healing of the wound. Further confirmation of collagen deposition is done by using Masson's trichrome stain, which stains specifically the collagen. It is observed that rats treated with the current formulation shows good deposition of Collagen as opposed to Standard and diseased control. This staining confirms the histology results.

Immunohistochemistry of (proliferation marker) Ki67 was done to check the cell proliferation at the wound site. Cell proliferation is observed to be more in animals treated with the current formulation as compared to standard and Diseased control, highlighting the proliferating activity of the current formulation

### ROS Analysis:

A series of recent studies have highlighted the important role of ROS in the wound healing process. They are required for efficient defense against invading pathogens. However, excessive production of ROS or impaired detoxification of these aggressive molecules causes oxidative stress, and this has been identified as an important feature in the pathogenesis of chronic, non-healing wounds. Therefore, a balance regulation of ROS production and detoxification is crucial for the normal repair process. In fact, the use of antioxidants like has been a recent and an effective strategy for therapeutic approaches to such disorders by inhibiting Lipid peroxidase (LPO).

Studies carried out under present formulation on the LPO status reveal that the present formulation possesses significant antioxidant activity, which helps to prevent oxidative damage and promote the wound closure process as shown in FIG. 9

### Preparation of Gel Formulation:

Spingosine-1-Phosphate (10 μM), Gallic acid (1 mg) and were added to 100 ml of Silver nanoparticles (SNP). Carbopol (0.5%) added to the solution with continuous stirring on a magnetic stirrer. The solution was neutralized by Triethanolamine. For base gel preparation 0.5% Carbopol-934 was added to 100 ml deionized water with continuous stirring, the solution was neutralized with Triethanolamine. The gels were stored at 4° C. till their further use.

The novel formulation is safe and proliferating to endothelial cells.

### EXAMPLES

### Example 1

Sphingosine-1-Phosphate (1 mM), Gallic acid (5 mg) and were added to 100 ml of Silver nanoparticles (SNP) 1100 ppm. Carbopol (0.5%) added to the solution with continuous stirring on a magnetic stirrer. The solution was neutralized by Triethanolamine. For base gel preparation 0.5% Carbopol-934 was added to 100 ml deionized water with continuous stirring, the solution was neutralized with Triethanolamine. The gels were stored at 4° C. till their further use.

### Example 2

Sphingosine-1-Phosphate (100 μM), Gallic acid (2.5 mg) and were added to 100 ml of Silver nanoparticles (SNP) 1500 ppm. Carbopol (0.5%) added to the solution with continuous stirring on a magnetic stirrer. The solution was neutralized by Triethanolamine. For base gel preparation 0.5% Carbopol-934 was added to 100 ml deionized water with continuous stirring, the solution was neutralized with Triethanolamine. The gels were stored at 4° C. till their further use.

### Antimicrobial Activity

Antimicrobial activity is checked against seven common wound pathogens by performing well plate method (FIG. 1). The data shows larger zone of inhibition when the formulation of the present invention applied as compared to silver nano-particles, Gallic acid, sphingosine-1-phosphate and collagen hydrolysates applied alone.

### Toxicity Data:

The toxicity of each component is checked on Human cell lines (Human umbilical cord endothelial cells). FIG. 2 represents adding silver nanoparticles up to 300 μg/mL there is more than 70% cell viability. Gallic acid has 100% human cell growth while treatment with as much as 250 μg/mL and Sphingosine-1-phosphate is not toxic in-fact it's proliferative at as low as 1 μM concentration. Further, all these components brought together in the novel formulation is safe (FIG. 2).

### Fibroblast Migration:

The fibroblast cells are grown in a well and a scarring is performed onto the cells followed by treatment with novel formulation solution. FIG. 3 represents that with novel formulation gap is filling fast as compared to control. When the data was quantitative and put upon a graph (FIG. 4), the difference between the novel formulation and control was significant.

### Gel Release Study:

Small discs of agar (1.5%) is made and added on top of 300 μl of deionized water to which 100 μl of the gel was added. The deionized water is collected at different time points and components concentration is estimated from the same.

### Antioxidant Assay:

The antioxidant used in the formulation is Gallic acid. The release of Gallic acidic measured by standard micro well plate assay (Modified McDonald method) and the plate was read at 690 nm. The gel formulation showed release of Gallic acid till 3^{rd} week. Release of Gallic acid on day 1 increased with increase in time. (FIG. 5)

### Excision Wound Model:

Rats were anesthetized with Xyalzine (22-24 mg/kg) and Ketamine (10-12 mg/kg, ip) and an area of about 330±10 mm² was marked on the dorsal side of the rat. The marked skin was cut carefully. During the study the wound areas were measured on 1 mm² graph paper on days 0, 3, 6, 9, 12, 15 and 18 for all groups. Change in wound area was measured regularly and the rate of wound contraction was calculated as given in the formula below. The wound healing of the test groups was calculated by comparing healed wound area on respective days with healed wound area of control group.

The percent wound contraction was measured after every four days for all groups and a percent wound closure graph was plotted. (FIG. 9)

Percent wound contraction=(Healed area)/(Initial wound area)×100

### Wound Healing

The in vivo results of novel formulation showed better wound contraction and healing as compared to standard control standard-gel and Disease control. The current formulation showed 5% more wound healing. (FIG. 13) Further, the wound healing is rapid by increasing the dose of the components. Histopathological analysis of the skin from all the groups showed no adverse reaction when novel formulation was used topically for the treatment of rats.

### BRIEF DESCRIPTION OF DRAWINGS

FIG. 1 is a graphical representation of antimicrobial activity of the novel formulation against the common infection causing bacteria

FIG. 2 is a representative of the toxicity of each component in the novel formulation on endothelial cells

FIG. 3 shows the fibroblast migration when the wound is administered with just lipid formulation, Sphingosine-1-phosphate (1 μM) and Sphingosine-1-phosphate (0.1 μM)

FIG. 4 shows the quantitative data of fibroblast migration against different concentrations of Sphingosine-1-phossphate and lipid formulation

FIG. 5 shows antioxidant assay of Gallic acid used in the formulation

FIG. 6 shows wound healing in natural conditions i.e. control

FIG. 7 shows wound healing after application of novel formulation

FIG. 8 shows wound closure percentage in vivo in excision wound model in rats

FIGS. 9 and 10 represents levels of Lipid peroxidation and Catalase in the wound area.

FIG. 11 is Photomicrographs of histology from wound site

### DETAILED DESCRIPTION OF THE DRAWINGS

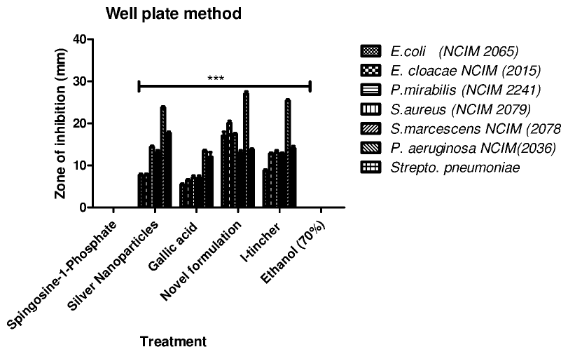

FIG. 1 further explains the antimicrobial activity of each of the components in the novel formulation along with novel formulation, Iodine Tincture and Ethanol against the common infection causing bacteria such as E.coli, E. cloacae, P. mirabilis, S. aureus, S. marcescens, P.aeruginosa, S.pneumoniae by using well plate method which shows larger zone of inhibition when treated with novel formulation as compared to single component such as to silver nano-particles, Gallic acid, sphingosine-1-phosphate and collagen hydrolysates

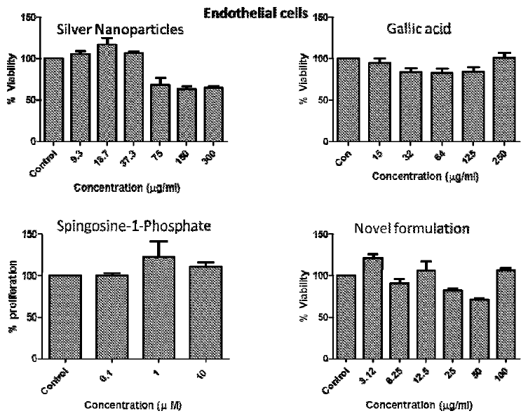

FIG. 2 further explains the toxicity of Silver nanoparticles on Human cell lines (Human umbilical cord endothelial cells) after adding individual components such as silver nanoparticles, Gallic acid and sphingosine-1-phoshpate and the novel formulation

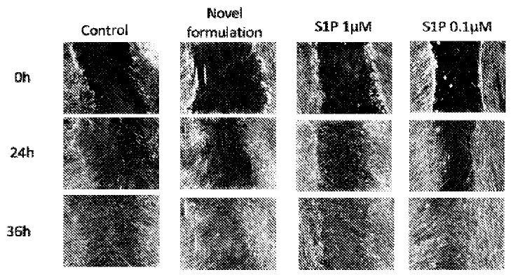

FIG. 3 explains fibroblast migration in the wound healing process after regular intervals of 24 hours to 36 hours after the onset of wound. When the wound is treated with the novel formulation the gap fills fast i.e. the fibroblast migration happens fast

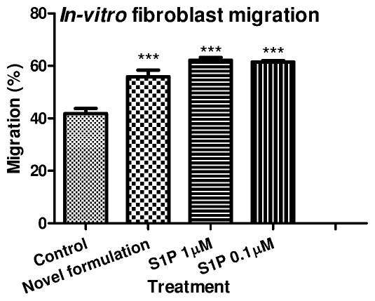

FIG. 4 explains the quantitative data represented in a graph showing the difference between the novel formulation and control was significant when S1P is added at 1 μM concentration which is responsible for fibroblast migration and also for proliferation

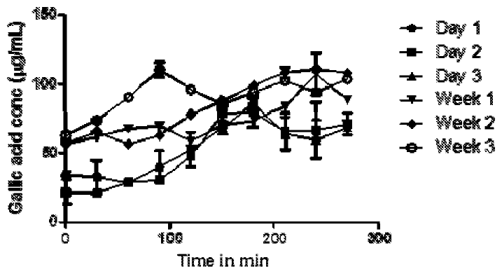

FIG. 5 further explains the release of Gallic acid as an indicator of stability of the gel over time. The release pattern of Gallic acid is not changing with time over 3 weeks. The Gallic acid release over the period of 300 min is increasing indicating a sustained release from the gel

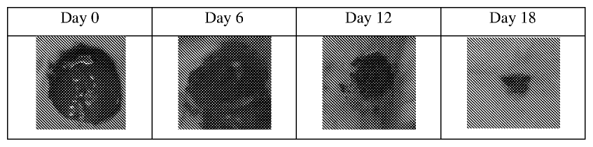

FIG. 6 represents the control rat wound healing, excision wound was formed and representative images were taken on 0, 6, 12 and 18 days. Wound healing is observed, however on the 16^{th} day a little scar observed.

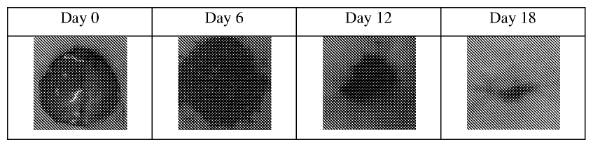

FIG. 7 represents wound closure in the test rat

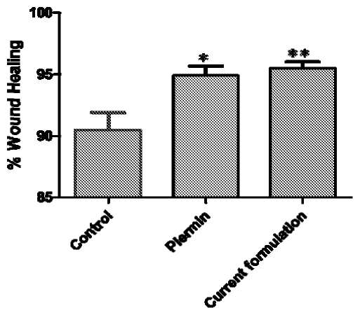

FIG. 8 Current formulation showed a significant increase in wound healing (**p<0.01) as compared to control. The wound healing results for standard gel Plermin used were comparable with the current formulation

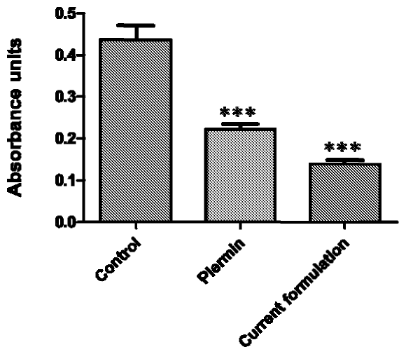

FIG. 9 shows the levels of LPO in rats treated with current formulation was lesser than the rats treated with standard gel. This justifies that the current formulation is effective against the oxidative stress generated during the wound healing procedure.

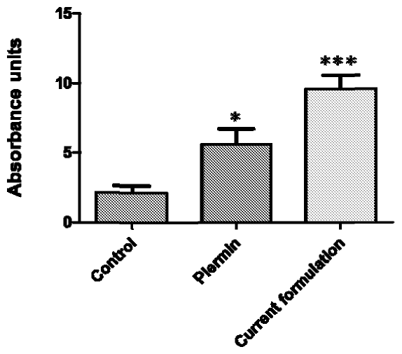

FIG. 10 show that the levels of catalase were increased significantly for current formulation treated groups

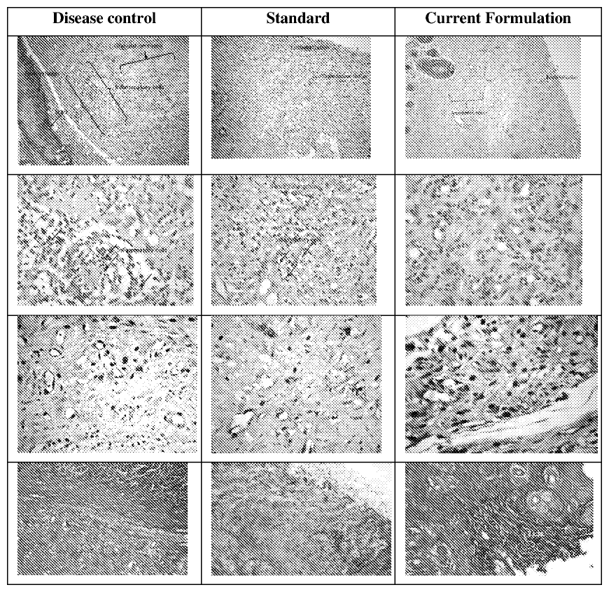

FIG. 11 shows that the inflammatory cells neutrophils and macrophages were more in diseased control as compared with the standard control and current formulation. The presence of inflammatory cells confirms prolong inflammation in control which delays wound healing. The presence of these cells shows an early stage of wound healing in diseased control rats while in the rats treated with the current formulation, inflammation has reduced indicating the progress of wound healing. Granulation tissue, proliferation of fibroblast cells and Collagen deposition is more in current formulation than in control and standard gel used (Panel 1 horizontal). The Collagen deposition is more compact in the group of rats treated with current formulation. In the control group the deposition is in patches (Panel 2 horizontal). Further confirmation of collagen deposition is done by using Masson's trichrome stain which stains specifically the collagen. In this staining it is found that rats treated with the current formulation shows good deposition of Collagen as compared with Standard and diseased control (Panel 3 horizontal). This staining confirms the histology results. Immunohistochemistry of proliferation marker Ki67 is done to check the cell proliferation at the wound site. It is observed to be more in animals treated with the current formulation as compared to standard and Diseased control, highlighting the proliferating activity of the current formulation (Panel 4 horizontal).

### Abbreviations Used:

S1P: Sphingosine-1-Phosphate
LPO: Lipid Peroxidase
ROS: Reactive oxygen species

### Advantages:

The novel formulation helps in rapid wound healing even at lower dosage
The novel formulation of present invention eliminates the repeated usage of antibiotics
There is no need of skilled personnel for treatment.
The novel formulation is cost effective and affordable.
There is no adverse reaction of the current formulation when applied topically.
The novel formulation helps the wound to heal holistically faster by addressing bacterial infection, ROS at wound site and angiogenesis

We claim:

1. A novel formulation for rapid wound healing and control of infection comprising:
Silver Nanoparticles as an antibacterial agent,
an antioxidant agent and;
Sphingosine-1-phosphate as an inducer of endothelial cell proliferation
wherein, the formulation is locally acting topical formulation.

2. A formulation for rapid wound healing and control of infection as claimed in claim 1, wherein said antioxidant agent is selected from Gallic acid, group of Vitamins, Bioflavonoid, Carotenoids, Hydroxycinnamates, Other natural antioxidants like Theaflavin, theaflavin-3-gallate, allicin, piperine, curcumin, Physiological antioxidants, Fungal antioxidants and synthetic antioxidants such as Cinnamic acid derivatives.

3. A formulation of claim 1, wherein concentration of silver nanoparticles is in the range of 0.8 mg/g-1.5 mg/g of gel

4. A formulation of claim 1, wherein the concentration of Gallic acid is in the range of 1 mg/g-5 mg/g of gel

5. A formulation of claim 1, wherein the concentration of Sphingosine-1-phosphate is in the range of 10 μM-1 mM/g of gel

6. A novel formulation for rapid wound healing and control of infection as claimed in claim 1, wherein the size of Silver nanoparticles is in the range of 10 nm to 100 nm

7. A method preparing the formulation as claimed in claim 1, comprising the steps of:
i. Adding 10 μM Spingosine-1-Phosphate and 1 mg Gallic Acid to 100 ml of Silver nanoparticles
ii. Adding 0.5% of carbopol to the above solution
iii. Continuously stirring the above solution on a magnetic stirrer and neutralizing the resultant solution with Triethanolamine

8. The formulation as claimed in claim 1, wherein the formulation can be in the form of a cream, gel, topical solution, patch, ointment, spray or lotion, patch or wipe.

9. Method of preparing the formulation as claimed in claim 8, is carried out at pH 7.0.
